(19) United States
(12) Patent Application Publication
(10) Pub. No.: US 2018/0228801 A1
(43) Pub. Date: Aug. 16, 2018

(54) Method for Enhancing Learning Ability and Memory of Patients with Alzheimer's Disease using Mocetinostat

(71) Applicant: National Taiwan Normal University, Taipei City (TW)

(72) Inventors: Hsiu-Mei Hsieh, Taipei City (TW); Hui-Chen Huang, Taipei City (TW); Hsin-Yu Huang, New Taipei City (TW)

(21) Appl. No.: 15/690,910

(22) Filed: Aug. 30, 2017

(30) Foreign Application Priority Data
Feb. 15, 2017 (TW) 106104951

(51) Int. Cl. A61K 31/506; A61K 47/46; A61K 9/00

(52) U.S. Cl. CPC A61K 31/506; A61K 9/0019; A61K 47/46

(57) ABSTRACT

A method for enhancing learning ability and memory of patients with Alzheimer's disease using mocetinostat is provided. The method includes administering an effective dose of mocetinostat into a subject in need. Based on the administration of mocetinostat, Aβ accumulation, Tau protein phosphorylation, and neuroinflammation can be reduced, and the level of synaptophysin and numbers of serotonergic neuron can be increased. Hence, the damage caused by the injection of oligomeric Aβ₂₅₋₃₅ within hippocampal CA1 is relieved. The method may be a potential solution for relieving the symptoms of anxiety and cognitive impairment.

### CROSS-REFERENCE TO RELATED APPLICATION

This application claims priority from Taiwan Patent Application No. 106104951, filed on Feb. 15, 2017 at the Taiwan Intellectual Property Office, the contents of which are hereby incorporated by reference in their entirety for all purposes.

### BACKGROUND OF THE INVENTION

### 1. Field of the Invention

The present invention relates to a method for enhancing learning ability and memory of patients with Alzheimer's disease using mocetinostat (MGCD0103).

### 2. Description of the Related Art

Dementia is one of the most common neurodegenerative diseases, and is actually a collective term covering several neurodegenerative diseases that have similar symptoms, wherein a large majority of cases are caused by Alzheimer's disease. Currently, there are about 24 million people around the world suffering from Alzheimer's disease, which increases by 4.6 million cases per year as the aging population increases. The World Health Organization (WHO) has estimated that Alzheimer's will affect 80 million people by the year 2040. Alzheimer's disease has a slow pathogenesis, and is a persistent neurological dysfunction which deteriorates over time. Early symptoms of Alzheimer's disease may include short term memory loss, whereas symptoms in later stages of Alzheimer's may include delirium, irritability, aggressive behavior, problems with language, disorientation (including easily getting lost), mood swings, loss of motivation, loss of long-term memory, not managing self-care and behavioral issues. Although how the disease progresses vary from person to person, in general, the life expectancy of a confirmed case is three to nine years.

The causes of Alzheimer's disease have not yet been fully understood. It has been found that amyloid plaques and Neurofibrillary Tangles (NFTs) build up around the neuron cells in the brain of Alzheimer's patients, and also that the nucleus basalis of Meynert degenerates, accompanied with a decreased level of acetylcholine neurotransmitter. The term NFTs refers to the seriously deformed tangled shape of neurons that are also found stacked in groups, as discerned by neuropathology. It is known that the formation of NFTs is related to hyperphosphorylation of the Tau protein, which causes aggregation of the Tau proteins and seems to be toxic to cells, thereby indirectly or directly damaging neurons.

Although various kinds of drugs have been developed for Alzheimer's disease, the largest predicament in the fight against Alzheimer's disease is that there is still lack of single effective therapeutic method. Histone deacetylase inhibitor (HDACi) plays an important role in the regulation of amyloid plaque, GSK3β, and Tau protein activity in patients with Alzheimer's disease. Uses of several HDACi-based drugs have been reported for the treatment of Alzheimer's disease and other neurodegenerative diseases, such as Huntington's disease and Parkinson's disease, in animal models. However, most HDACi-based drugs relate to a non-selective inhibitor. Since their therapeutic mechanisms are not yet clear, different kinds of HDACi drugs are combined as a strategy for the treatment of such diseases.

### SUMMARY OF THE INVENTION

Given the above described limitations, the purpose of the present invention is to provide a method for enhancing learning ability and memory of patients with Alzheimer's disease using mocetinostat (MGCD0103). The HDACi mocetinostat may provide relief from anxiety, improve short-term and long-term memory, reduce the accumulation of β-amyloid (Aβ), the hyperphosphorylation of tau protein, and neuroinflammation, and increase the numbers of noradrenergic neurons and the expression level of synaptophysin against damage-induced by oligomeric Aβ₂₅₋₃₅, and further improve the learning and memory ability of a subject with AD.

According to an aspect of the present invention, a method for enhancing learning ability and memory of patients with Alzheimer's disease using mocetinostat is provided, wherein HDACi mocetinostat is administrated to a subject in need at an effective dose.

Preferably, the effective dose of mocetinostat may be 0.01˜2 mg per kilogram of body weight.

Preferably, mocetinostat may be used in combination with saline and pharmaceutically acceptable excipients.

Preferably, the ratio of saline and the pharmaceutically acceptable excipients may be 3˜8:1.

Preferably, the pharmaceutically acceptable excipients may be Kolliphor®.

Preferably, the administration route may include via oral, intramuscular, subcutaneous or brain administration.

Preferably, mocetinostat may reduce the accumulation of β-amyloid (Aβ), the hyperphosphorylation of tau protein, and neuroinflammation, and may increase the numbers of noradrenergic neurons and the expression of synaptophysin protein.

The method for enhancing learning ability and memory of patients with Alzheimer's disease using mocetinostat according to the present invention may include the following advantages:

(1) According to the method of the present invention, administrating mocetinostat may significantly improve neuron viability and length of neurites.

(2) According to the method of the present invention, administrating mocetinostat may provide relief from anxiety and from deterioration of spatial learning and memory abilities caused by oligomeric Aβ₂₅₋₃₅.

(3) According to the method of the present invention, administrating mocetinostat may reduce the accumulation of β-amyloid (Aβ), the hyperphosphorylation of tau protein, and neuroinflammation, and may increase the numbers of serotonergic neurons and the expression of synaptophysin protein so that it may become potential medication for treating anxiety and memory dysfunction.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A to 1D show the in vitro culturing results of primary hippocampus neuron cells treated by low and high dose of MGCD0103.

FIG. 1A is a timing diagram illustrating the progress of cell culturing and treatment of MGCD0103; FIG. 1B shows results of immune-fluorescent staining; FIG. 1C is a bar chart illustrating relative numbers of cells expressing the Neu N⁺ protein; and FIG. 1D is a bar chart illustrating relative lengths of neurites. * represents a comparison with control group; # represents a comparison with the group treating Aβ₂₅₋₃₅ alone.

FIG. 2 shows a bar chart illustrating the viability results affected by treating the primary hippocampus neurons with MGCD0103.

FIG. 3 illustrates an in vivo experiment process, wherein MGCD0103 is administrated to a subject by intraperitoneal injection (i.p.) at a dose of 0.01 mg/kg (low dose) and 0.5 mg/kg (high dose).

FIGS. 4A to 4D show the analysis results of the Open Field Test, Elevated Plus Maze (EPM) Test and Y-maze Test. FIG. 4A shows spontaneous exercise ability of mice in the Open Field Test; FIG. 4B shows anxiety results of the mice in the Open Field Test; FIG. 4C shows the mice's overall time spent in the open arms of the Elevated Plus Maze; and FIG. 4D shows analysis results of spontaneous alternation rate of the mice in the Y-Maze Test. * represents a comparison with group treated with saline; # represents a comparison with the group treated with Aβ₂₅₋₃₅ alone.

FIGS. 5A to 5D show the results concerning the Morris Water Maze (MWM). FIG. 5A shows the swimming velocity of the mice; FIG. 5B shows the learning curve with a training period of 4 days, wherein the symbols represent: the normal mice (◯), the mice of which the hippocampal CA1 injected with oligomeric Aβ₂₅₋₃₅ (●), the mice with hippocampal CA1 injected with oligomeric Aβ₂₅₋₃₅ and treated with a low dose MGCD0103 (▪), the mice with hippocampal CA1 injected with oligomeric Aβ₂₅₋₃₅ and treated with a high dose MGCD0103 (□), the mice with hippocampal CA1 injected with saline and treated with a low dose MGCD0103 (▾); and the mice with hippocampal CA1 injected with saline and treated with a high dose MGCD0103 (Δ); FIG. 5C shows the analysis results of learning ability on day 5; and FIG. 5D shows the results of the overall time that the mice spend at the platform of the original quadrant after removing the platform on day 6. * represents a comparison with the group treated with saline; # represents a comparison with the group treated with Aβ₂₅₋₃₅ alone.

FIGS. 6A to 6C shows the analysis results of the acetylation level of tissue proteins H3 and alpha-tubulin. FIG. 6A shows the western blot staining of acetylated tissue proteins H3 and alpha-tubulin; FIG. 6B shows the quantitative results of acetylated tissue proteins H3; FIG. 6C shows the quantitative results of acetylated alpha-tubulin. * represents a comparison with the group treating saline; # represents a comparison with the group treating Aβ₂₅₋₃₅ alone.

FIGS. 7A to 7C show the analysis results of the proteins related to a neuron synapse. FIG. 7A shows the western blot staining of synaptophysin and PSD95 proteins; FIG. 7B shows the quantitative results of the synaptophysin protein; and FIG. 7C shows the quantitative results of the PSD95 protein. * represents a comparison with the group treated with saline; # represents a comparison with the group treated with Aβ₂₅₋₃₅ alone.

FIGS. 8A to 8C show the analysis results of proteins related to tau protein phosphorylation. FIG. 8A is a western blot staining of the proteins related to tau protein phosphorylation; and FIGS. 8B and 8C show bar charts illustrating quantitative results of the proteins related to tau protein phosphorylation. * represents a comparison with the group treated with saline; # represents a comparison with the group treated with Aβ₂₅₋₃₅ alone.

FIGS. 9A to 9C show the analysis results of enzymes related to tau protein phosphorylation. FIG. 9A is a western blot staining of the proteins related to pCDK and pERK protein phosphorylation; and FIGS. 9B and 9C show quantitative results of the proteins. * represents a comparison with the group treated with saline; # represents a comparison with the group treated with Aβ₂₅₋₃₅ alone.

FIGS. 10A to 10C show immunochemical analysis results of the tau protein phosphorylation at the S202 site in hippocampal CA1 and basolateral nucleus amygdala (BLA) region. FIG. 10A shows the immunohostochemical staining results of the tissue slice analyzing the tau protein phosphorylation at the S202 site; FIG. 10B shows the quantitative results of the hippocampal CA1 region; and FIG. 10C shows the quantitative results of the BLA region. * represents a comparison with the control group; # represents a comparison with the group treating Aβ₂₅₋₃₅ alone.

FIGS. 11A and 11B show immunochemical analysis results of Aβ accumulation in hippocampal CA1 region. FIG. 11A shows the immunohistocehmical staining results of the 6E10; and FIG. 11B shows the quantitative results thereof. * represents a comparison with the group treated with saline; # represents a comparison with the group treated with Aβ₂₅₋₃₅ alone.

FIGS. 12A to 12D show the analysis results of the proteins related to the formation and removal of Aβ accumulation. FIG. 12A shows the western blot staining of BACE1, IDE and NEP protein; FIG. 12B shows the quantitative result of the BACE1 protein; FIG. 12C shows the quantitative result of the IDE protein; FIG. 12D shows the quantitative results of the NEP protein. * represents a comparison with the group treated with saline; # represents a comparison with the group treated with Aβ₂₅₋₃₅ alone.

FIG. 13A to 13C shows the immunochemical analysis results of glial cells in hippocampus. FIG. 13A shows the immunohistochemical staining results of astrocyte and activated microglia; FIG. 13B shows a bar chart illustrating the quantitative results of astrocyte; and FIG. 13C shows a bar chart illustrating the quantitative results of activated microglia. * represents a comparison with the group treated with saline; # represents a comparison with the group treated with Aβ₂₅₋₃₅ alone.

FIG. 14 shows the immunochemical staining results of cholinergic neurons, serotonergic neurons and adrenergic neurons.

### DESCRIPTION OF THE PREFERRED EMBODIMENTS

The present invention will be described in detail along with preferable embodiments and the drawings. It is to be noted that the experimental data disclosed in the following examples are intended to be illustrative of the technical features of the present invention and are not intended to limit the aspect in which they may be implemented.

### Definitions

Hereinafter, when terms such as “about” or “approximately” are used in combination with a measurable value as a variable, they refer to the assigned value of the variable, or a range of values within an experimental error (for example, the level of confidence of the average=95%), or the maximum value within all of the values that are smaller than 10% difference from the assigned value.

The term “administration” refers to importing a material into a subject, for instance, MGDC0103 is administrated into a subject by at least one route including: oral administration and non-oral administration (e.g. subcutaneous, intramuscular, transdermal, intradermal, intraperitoneal, intraocular and intravenous injections).

The term “subject” refers to any mammal with a potential need for being administered with the composition of the present invention, including: primates, rodents, pets, laboratory animals and domesticated animals. For example, this may include, but is not limited to, monkeys, humans, swine, cattle, sheep, goats, horses, mice, rats, guinea pigs, hamsters, rabbits, felines, and canines. Preferably, the subject in need is a mouse or a human.

The drug “mocetinostat” used in the present invention (hereinafter, also called “MGCD0103”) refers to an isoform-selective histone deacetylase inhibitor (HDACi), which has the chemical name “N-(2-Aminophenyl)-4-[[(4-pyridin-3-ylpyrimidin-2-yl)amino]methyl] benzamide” with the structure labeled as Formula 1 and shown as follows:

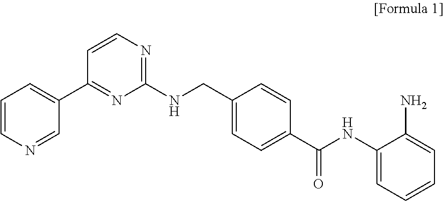 [SMILES: Nc1ccccc1NC(=O)c1ccc(CNc2nccc(-c3cccnc3)n2)cc1]

The term “prodrug” used in the present invention refers to a drug with a pharmacologically inactive form or low activity form with respect to an organism (e.g. a human), wherein the drug may be converted within the body into an active form by, for example, the body's metabolism. The conversion of the prodrug into an active form is not particularly limited and includes any chemical and/or physical changes that occur after administrating the prodrug, e.g., the prodrug releases the active moiety (especially the cell growth inhibitor) at an active site.

The term “solution” used in the present invention refers to a variable composition formed by a solute and a solvent. Such solvents for the purposes of the present invention may not interfere with the biological activity of the solute. Examples of suitable solvents include, but are not limited to, water, methanol, ethanol, acetic acid and DMSO (dimethyl sulfoxide). Preferably, the solvent is a pharmaceutically acceptable solvent. Examples of suitable pharmaceutically acceptable solvents include, but are not limited to, water, ethanol, acetic acid and DMSO.

The “pharmaceutically acceptable excipient” used in the present invention refers to any component which is not MGCD0103. It is selected from all common excipients known by a person skilled in the art depending on the required pharmaceutical form and on the manner of administration.

In an embodiment of the present invention, the pharmaceutically acceptable excipient may be a lipophilic excipient, a filler, a wetting agent, an adhesive agent, or a disintegrant, but is not limited thereto. Commonly used surfactants such as Tweens or Spans, or emulsifiers or bioavailability enhancers commonly used in manufacturing pharmaceutically acceptable solid, liquid, or other pharmaceutical forms may also be used, for example, as an excipient for the purpose of drug preparation. If necessary, sweeteners, flavoring agents, or coloring agents may be added.

The lipophilic excipients may be glyceryl stearate, palmitate/glyceryl stearate and glyceryl behenate, hydrogenated vegetable oils and derivatives thereof, plants and animals waxes and derivatives thereof, hydrogenated castor oil and derivatives thereof, and cetyl esters and preferably Kolliphor® (available from Sigma, C5135, USA).

The filler may be one or more substances selected from, but not limited to the group consisting of lactose, sugar, starches, modified starches, mannitol, sorbitol, inorganic salts, cellulose derivatives (e.g. microcrystalline cellulose, cellulose), calcium sulfate, xylitol, lactitol, and mixtures thereof.

The wetting agent may be one or more substances selected from, but not limited to the group consisting of distilled water, ethanol, starch paste, and mixtures thereof.

The adhesive agent may be one or more substances selected from, but not limited to the group consisting of acacia, gelatin, tragacanth, dextrin, polyvinylpyrrolidone, starch and derivatives thereof, sodium alginate, sorbitol, syrup, hypromellose, methyl cellulose, hydroxypropyl cellulose, hydroxyethyl cellulose, ethyl cellulose, sodium carboxymethyl cellulose, calcium carboxymethyl cellulose, glucose, polymethacrylates, and mixtures thereof.

The disintegrant may be one or more substances selected from, but not limited to the group consisting of crosscarmellose sodium, crospovidone, polyvinylpyrrolidone, sodium starch glycollate, corn starch, microcrystalline cellulose, hydroxypropyl methylcellulose, hydroxypropyl cellulose, and mixtures thereof.

In particular, preparations of various solutions for treating or for use as control groups in embodiments of the present invention are described as follows.

In some embodiments, oligomeric Aβ₂₅₋₃₅ for in vitro use is prepared by dissolving Aβ₂₅₋₃₅ (SigmaSI-A4559, USA) in water under 37° C. and leaving to stand for 4 days. Further, oligomeric Aβ₂₅₋₃₅ for in vivo use is prepared by dissolving said Aβ₂₅₋₃₅ in saline at 37° C. and leaving to stand for 7 days.

In some embodiments, MGCD0103 for in vivo use is prepared by dissolving MGCD0103 in DMSO (40 mg/mL), thereby dissolving in a mixture of saline and Kolliphor® during intraperitoneal (i.p.) injection, wherein the ratio of the saline and the Kolliphor® is about 3˜8:1, preferably about 4˜6:1, most preferably about 4:1.

In some embodiments of the present invention, the methods for culturing the mouse hippocampus primary neurons and performing the immunochemical tissue staining analysis thereof are described in detail as follows.

Methods of Culturing the Mouse Hippocampus Primary Neurons

The method of culturing mouse primary hippocampus neurons follow from a modified method from the prior art (Seibenhener and Wooten, 2012), including: obtaining a 16-18 day old embryo from a C57BL/6J strain pregnant female mouse after euthanasia, obtaining hippocampus tissue of the embryo and digesting the tissue with 0.05% trypsin at 37° C. for 15 minutes, and seeding 3×10⁴ cells per well in a 48-well plate coated with Poly-L-lysine (100 μg/mL); wherein the components of the medium includes: Neurobasal Medium® (Gibco™; ThermoFisher Scientific, USA) adding 2% of B-27® Additive (Gibco™; ThermoFisher), 0.5 mM of glutamine (Gibco™; ThermoFisher), 25 μM of glutamate (Sigma-Aldrich, USA), 20 unit/mL of penicillin/streptomycin solution (Gibco™; ThermoFisher Scientific, USA), 1 mM of 4-(2-hydroxyethyl)-1-piperazineethanesulfonic acid) (HEPES) (Sigma-Aldrich), and 1% of heat inactivated Donor Horse Serum (Gibco™; ThermoFisher). Primary hippocampus neurons are cultured in an incubator in an environment of 37° C. and 5% CO₂.

In Vitro Drug Treatment

In some embodiments, experiments should be performed when said cells are in an Alzheimer's disease state. Hence, the inventor treats said primary hippocampus neurons with said oligomeric Aβ₂₅₋₃₅ in order to reduce the numbers of neurons and branches, and length of neurite, so as to simulate a pathological state of Alzheimer's disease. Further, 50 μM of oligomeric Aβ₂₅₋₃₅ are applied for 1 hour, and then a high dose (70 nM) and low dose (35 nm) of MGCD0103 is applied for 48 hours on day 9. Finally, the cells are collected for immune-fluorescent staining.

Immune-Fluorescence Staining Analysis

The collected cells are analyzed with immune-fluorescence staining. First, fixing the cells with 4% of paraformaldehyde (PFA) (Sigma-Aldrich) for 30 minutes; rinsing the cells three times with PBST with a time interval of 10 minutes so as to remove remaining PFA; blocking the cells with 10% fetal bovine serum (FBS) for 2 hours; adding NeuN (1:1000; Milipore, USA) and MAP2 (1:1000; Milipore, USA) primary antibodies and allowing reaction at 4° C. for 16 hours; then allowing reaction with secondary antibodies at 37° C. for 2 hours; finally, staining nucleus with 4′,6-diamino-2-phenylindole (DAPI) (Sigma-Aldrich) and analyzing to obtain values such as numbers of neurons, lengths of synapses, and numbers of branches with a High Content Micro-Imaging Acquisition and Screening System and MetaXpress (purchased from Molecular Devices).

Cell Viability Analysis (MTT Assay)

(3-(4,5-Dimethylthiazol-2-yl)-2,5-diphenyltetrazolium bromide, MTT) (Sigma, USA) has an original color of yellow, which is able to carry out a reducing reaction by a succinate dehydrogenase of mitochondria within living cells that breaks the tetrazolium ring thereof so as to form formazan, which has a violet color. Thereby, DMSO is added in order to dissolve the violet crystal. Finally, the cell viability may be analyzed by detecting the changes of violet color (O.D. 570) by a spectrophotometer. In the present invention, primary hippocampus neurons are treated with various doses of MGCD0103, and the cell viability is measured after 48 hours.

In some embodiments of the present invention, the subject in need, the administrating methods, evaluating methods of the behavior of the subject, and the pathological analysis methods used in the embodiments of the present invention are described in details as follows.

In Vivo Experiments

In embodiments of the present invention, the subject in need for in vivo experiments may be mice.

As mentioned above, the mice may be C57BL/6J strain, 8 week old pregnant female mice and 12 week old male mice (purchased from National Laboratory Animal Center, Taiwan). The breeding environment is configured as 20-25° C., 60% relative humidity and 12 hours circadian rhythm. All experiments were performed from 7 am to 7 pm, and comply with the provision pursuant to the regulations stipulated by the Committee of Care and Use of Laboratory Animals of National Taiwan Normal University.

Drug Treatment of In Vivo Experiments

MGCD0103 may possess or not possess one or more effective doses which are administrated into a subject in accordance with a specific time interval. The administrating frequency may vary depending on any of various factors, such as severity of the symptoms, the protection level that the subject needs, and whether the purpose is for prevention or therapy. For instance, in one embodiment, MGCD0103 may be administrated once a day.

Further, in some embodiments of the present invention, C57BL/6J strain male mice (12 week old) are used as a subject in need. After mice are allowed to adapt to the environment for 6 days, mice are anesthetized with Avertin (0.4 g/kg; purchased from Sigma). The mice are fixed on a stereotactic apparatus for operation, followed by injecting oligomeric Aβ₂₅₋₃₅ (10 nM, 3 μL) into bilateral hippocampal CA1 (AP: −0.23 mm relative to the bregma; ML: ±0.2 mm relative to the midline; DV: −0.15 mm relative to the skull) on day 7. MGCD0103 and equal volume of vehicle (saline) are intraperitoneal (i.p.) injected once a day from day 8, the day after said operation, for 29 days. On day 24, the Open Field Test is performed; on day 26, the EPM is performed; on day 28, the Y-maze is performed; on days 30 to 36, MWM is performed; and on day 37, mice are euthanized for pathological analysis.

Accordingly, the dose of the administrated MGCD0103 is about 0.01˜2 mg/kg, preferably about 0.5˜1.5 mg/kg, more preferably 0.5˜1 mg/kg. When administrating a dose larger than 2 mg/kg into a subject, a problem of cell toxicity may occur. When administrating a dose smaller than 0.01 mg/kg into a subject, the result shown is that it is ineffective for improving short term memory and relieving anxious behaviors.

Open Field Test (OFT)

In an embodiment, OFT is performed to said mice. The mice are placed in a central area of a white box (30 cm×30 cm×30 cm). Then, mice are allowed spontaneously walk for 10 minutes, and the time that mice spend at the central area (15 cm×15 cm×15 cm) in the first 5 minutes is recorded. Since mice tend to spend time at a peripheral area of an open field when anxious, observing the time spent in the central zone there by the mice will give an indication of the level of anxiety of the mice. In addition, counting the total distance that the mice have moved in the last 5 minutes may determine an index of spontaneous exercise ability of the mice. After finishing the test for each mouse, the box was wiped with 70% and 30% (v/v) of ethanol solution to remove remaining odor in order to avoid affecting other test results.

Elevated Plus Maze Test

By means of observing the mice exploring an unfamiliar environment, and the contradictory and conflicting behavior caused by fear of the animal of highly hanging arms of the maze, the anxiety level of the animal can be determined. The elevated plus maze is arranged with two relative open arms (30 cm×5 cm) and two relative enclosed arms (30 cm×5 cm×15 cm) connected at a central area (10 cm×10 cm), wherein the material thereof is matte acrylic which may have its odor easily removed by ethanol. Each mouse is placed at the central area facing the open arms and is allowed to freely and spontaneously explore for 5 minutes. After finishing the test for each mouse, the box was wiped with 70% and 30% (v/v) of ethanol to remove remaining odor in order to avoid affecting other test results. The total time spent at the open arms for each mouse was recorded by a video tracking system (EthoVision-XT, Noldus).

Y-Maze Activity Test

Y-Maze Activity Test is a test that takes advantage of the characteristic that mice tend to explore an unfamiliar environment, so that the short-term spatial memory of mice can be measured by means of a Y-maze module arranged with three arms (35 cm×5 cm×20 cm) formed of white acrylic. The mice are placed in the middle of the three arms of the Y-Maze, and the mice are allowed to freely and spontaneously explore for 8 minutes, wherein one count is recorded when four limbs of the mice completely enter any one of the three arms. The formula is described as follows: the spontaneous alternative rate=number of times any one of three arms are entered (without counting repeat entries)×100/(total number of times any arm has been entered−2).

Morris Water Maze (MWM) Memory Test

MWM is a test for observing spatial learning and memory of mice by placing a platform in a wide pool. Since the mice do not like to spend time in water, and also it being hard for the mice to swim, the mice will instinctively try to find a place to rest (the platform) while in water. The behavior of finding the platform relates to a complex memory process within the brain, including 1) collecting the visual information (such as shape information of rectangles, circles and triangles) with respect to spatial positioning, and 2) processing, sorting, memorizing, fixing and recalling said information. In particular, the mice are placed in a pool filled with milky white non-toxic advertising pigment (used to make the water become opaque to hide the platform so that the mice may not know the position of the platform at the beginning), and are allowed to explore in search of the platform (which is fixed in a predetermined quadrant) underwater. The test is split into several phases as follows: 1) exploration phase: Place the mouse in the water and leave it there for 1 minute. If the mouse cannot find the platform in time, then move the mouse to the platform and leave there for 20 seconds. Then, place the mouse at a dry location and allow to rest until the next experiment; 2) acceptance phase: place the mouse into the water maze at four specific positions in turn in order to test whether the mouse can find the platform or not. Such training is repeated four times a day and continued for 4 days (each mouse is trained a total of 16 times). After 4 days of training, the learning ability of the mouse is measured in testing. After 24 hours of last testing trial, platform was removed and the mouse to freely swim in the pool and observe whether the mouse remembers the position of the platform or not (long-term spatial memory test). The swimming path is recorded by a CCD camera and analyzed with an image tracking system (EthoVision-XT).

Immunohistochemical Staining of Tissue Slices

The inventor collects the brain tissue by perfusion followed by fixing and dehydrating, then, 30 μm frozen sections are obtained by a freezing microtome (CMS3050S, Leica). The section is washed three times with PBS for 10 minutes each time so as to remove mounting gel; then, endogenous peroxidase is removed with 3% H₂O₂. Next, non-specific antigen is destroyed by applying a blocking solution for 1 hour, then primary antobodies (6E10, pS202Tau, ChAT, 5-HT, TH, GFAP, Ibal) are added and allowed to react for 12 hours. Then, secondary antigen (diluted 200 times in the blocking solution, Vector Laboratories, USA) is added and allowed to react for 1 hour. After that, avidin-biotin complex (ABC) is detected 1 hour after staining. Finally, colorate with a DAB-kit (DAB: diaminobenzidine; Vector Laboratories, USA). After all sections have been stained, they are then fixed on a slide, dried, dehydrated, mounted, and photographed for quantity (performed by Image Pro Plus, Meida Cyberetics, USA).

Statistical Methods

In the aforementioned embodiment, the results of two groups are compared by an independent sample t-test. Results of three or more groups are compared by a one-way ANOVA test, and post hoc tests are carried out using LSD (SPSS version 20; Illinois, USA). All of the results are indicated by Mean±SEM. Furthermore, p<0.05 is the measure used for statistical significance.

The embodiments of the present invention disclosed above have been implemented and the results are stated below. According to the results, the purposes, features and advantages of the present invention are easily realized. A person skilled in the art will understand that the results do not limit the scope of the present invention. Further, the reasonable error of the results should be presented when repeating.

Results of In Vitro Experiments

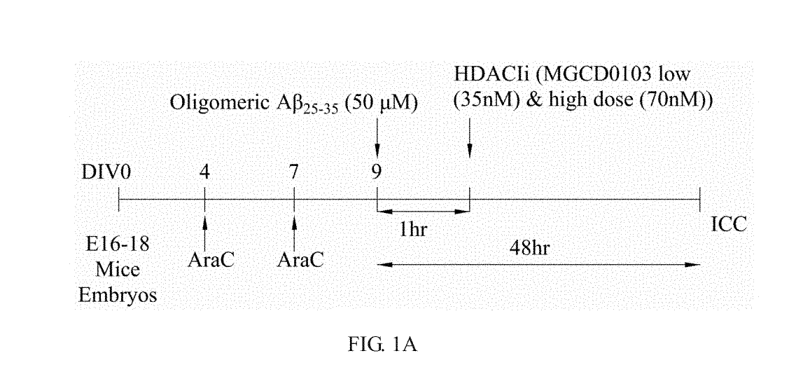

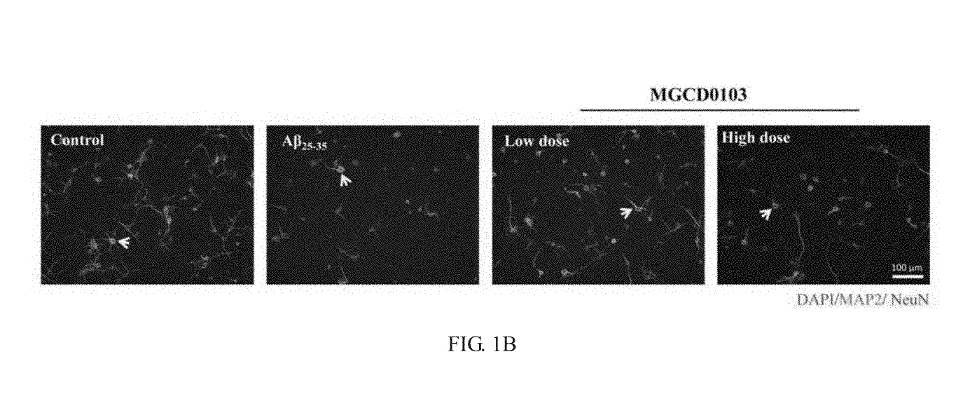

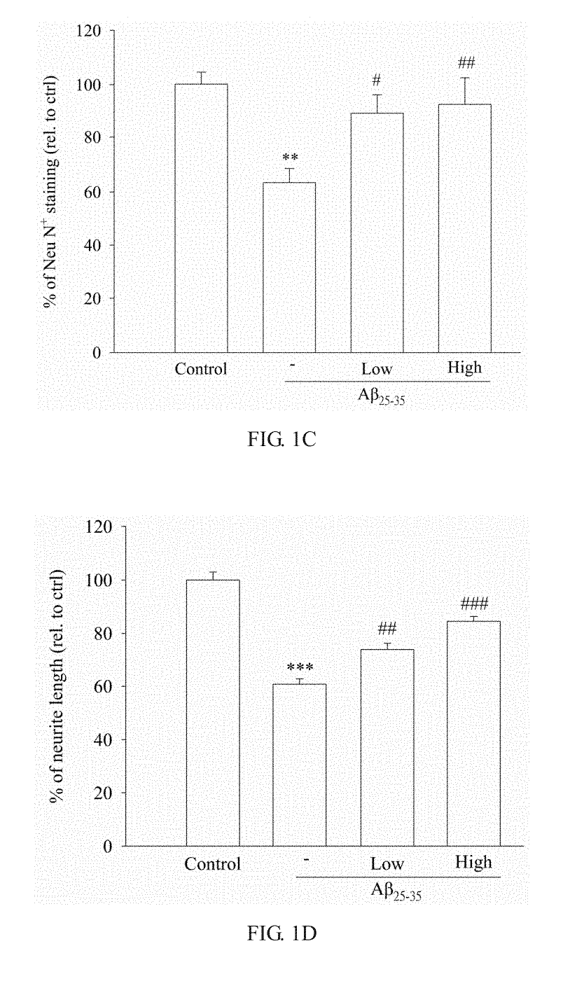

As shown in FIG. 1B to 1D, the results of neuron cells treated with MGCD0103 show that the numbers of neuron cells (FIGS. 1B and 1C) (counts of red stained region) and the lengths of neurites (FIGS. 1B and 1D) (measured by green stained region) may be significantly improved in the group treated with a high dose of MGCD0103 in comparison with the group treated with Aβ₂₅₋₃₅. Whereas, the numbers of neuron cells (FIGS. 1B and 1C) and the lengths of neurites (FIGS. 1B and 1D) may also be significantly improved in the group treated with a low dose (35 nM) of MGCD0103 in comparison with the group treated with Aβ₂₅₋₃₅. That is, the results show that MGCD0103 has a neuron protecting effect, which provides relief to the neuron cells damaged by Aβ₂₅₋₃₅.

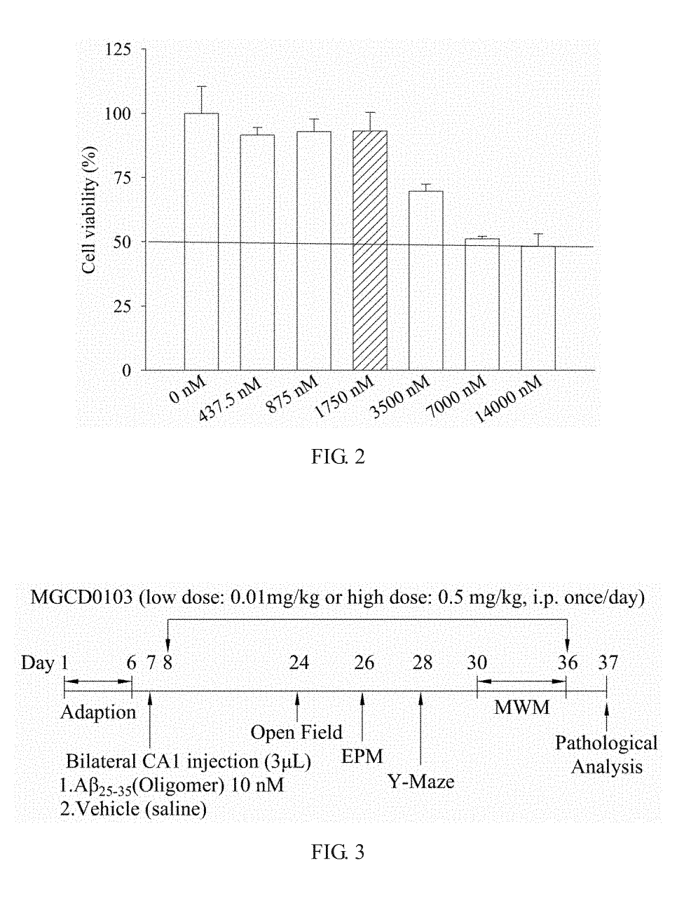

However, considering that most of HDACi passes through the blood-brain barrier (BBB) with a very low efficiency, doses of 0.01 and 0.05 mg/kg as the low and high doses for in vivo experiments are used according to some references (Pajouhesh and Lenz 2005; Boumber, Younes et al. 2011). To confirm that this dose (0.5 mg/kg) is still within the IC₅₀ range, the primary hippocampus neuron cells may be treated with MGCD0103 on day 9, and cell viability may then be assessed. As shown in FIG. 2, it was found that the IC₅₀ dose of MGCD0103 is about 7000 nM (equivalent to 2 mg/kg for in vivo) according to the cell viability results. Whereas the doses used in the present invention are 0.01 mg/kg (equivalent to 35 nM for in vitro) and 0.5 mg/kg (equivalent to 1750 nm for in vitro). Hence, the selected dose in the present invention is far less than the IC₅₀ dose.

Results of In Vivo Experiments

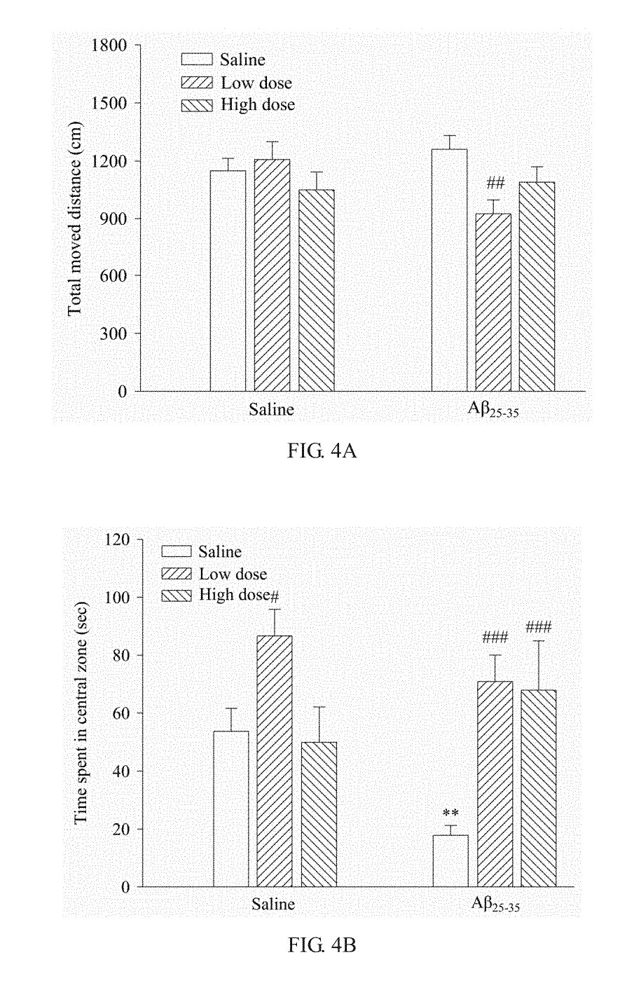

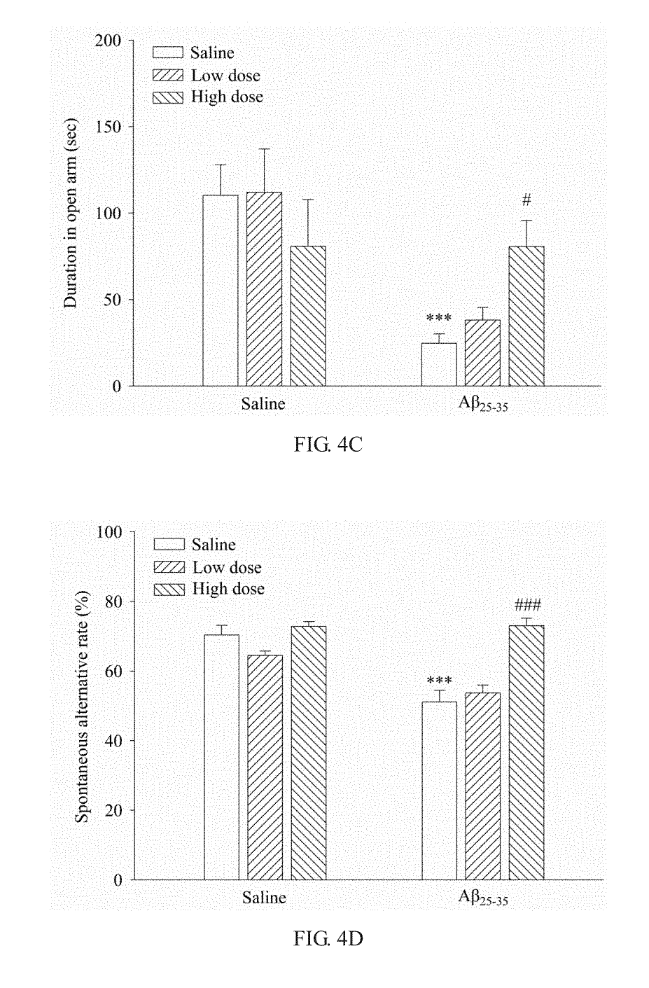

According to the in vitro experiments stated above, it has been shown as a brief conclusion that treating an Alzheimer's disease cell culture with MGCD0103 may protect neurons effectively. Hence, the inventor further administrates MGCD0103 into living mice in order to test whether the composition of the present invention may improve the cognitive ability of an Alzheimer's disease patient or not. Subsequently, the inventor further observes the effect of MGCD0103 on anxiety levels and short-term memory in mice affected by oligomeric Aβ₂₅₋₃₅. Referring to FIGS. 4A to 4D, the results compare the total moving distance of the mice in the present OF, EPM test, and Y-Maze test. It has been shown that the group treated with low dose MGCD0103 has a reduced mice moving total distance in comparison with the group treated with saline; and the remaining groups do not show a significant difference (FIG. 4A), which shows that treating with a high dose MGCD0103 does not influence the spontaneous exercise ability of mice. In addition, the inventor has also found that the mice injected with oligomeric Aβ₂₅₋₃₅ within the bilateral hippocampal CA1 spends less time in the central area than the mice injected with saline (FIG. 4B). This result shows that oligomeric Aβ₂₅₋₃₅ increases the anxiety levels of mice, whereas continuously administrating low and high doses of MGCD0103 both significantly increase the time mice spend at the central area (FIG. 4B). Such results show that treating with MGCD0103 may provide relief from increased level of anxiety in animals. In addition, the EPM test is another test for levels of anxiety in the mice, which utilizes the intrinsic acrophobia of the mice. It is performed by recording the time they spend in the open arm, wherein the longer the mice spend there indicates a lower anxiety level. As shown in FIG. 4C, the group injected with oligomeric Aβ₂₅₋₃₅ has significantly reduced time spent in the open arm, whereas that of the group treated with high dose of MGCD0103 is significantly increased. According to the results, administrating the high dose MGCD0103 of the present invention may significantly provide relief from the anxiety symptoms caused by oligomeric Aβ₂₅₋₃₅.

Further, the influence caused by treating with MGCD0103 against the deterioration in short-term memory ability caused by oligomeric Aβ₂₅₋₃₅ is observed. As shown in FIG. 4D, by calculating the spontaneous alternation rate according to the movement of the mice in the three arms of the Y-maze, the short term memory ability of the mice in each group may be evaluated. According to the test results, it was found that the spontaneous alternation rate of the mice injected with oligomeric Aβ₂₅₋₃₅ is significantly less in comparison with the mice treated with saline, that is, oligomeric Aβ₂₅₋₃₅ causes the deterioration in short-term memory ability. However, after administrating the high dose of MGCD0103, it was found that the spontaneous alternation rate had significantly increased. This means that the high dose MGCD0103 may provide significantly relief from deterioration in short term memory ability caused by oligomeric Aβ₂₅₋₃₅.

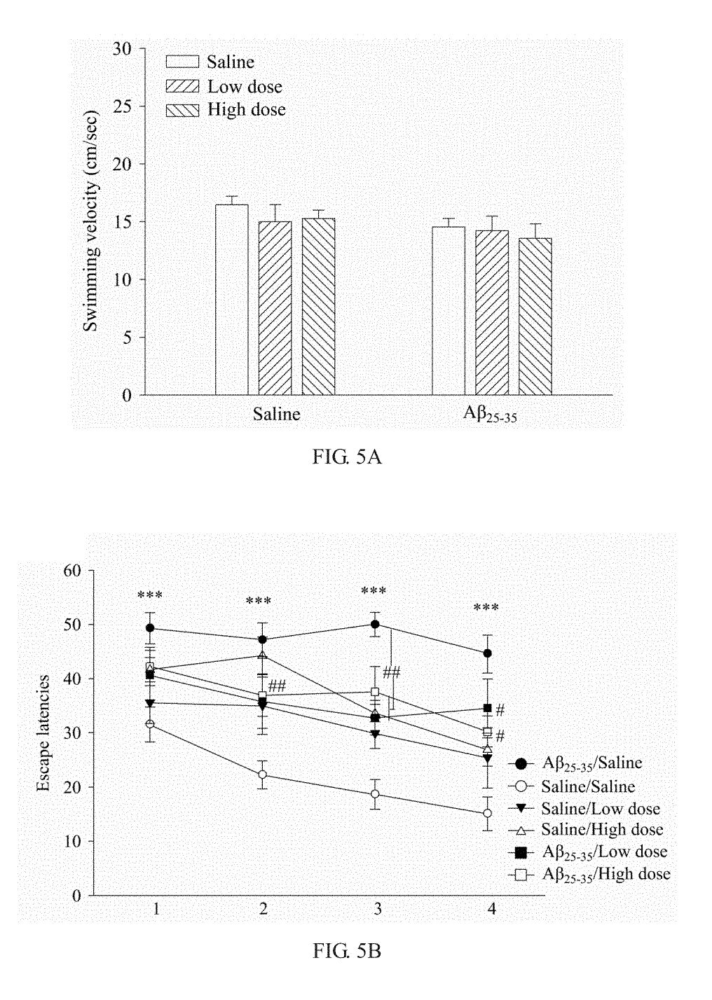

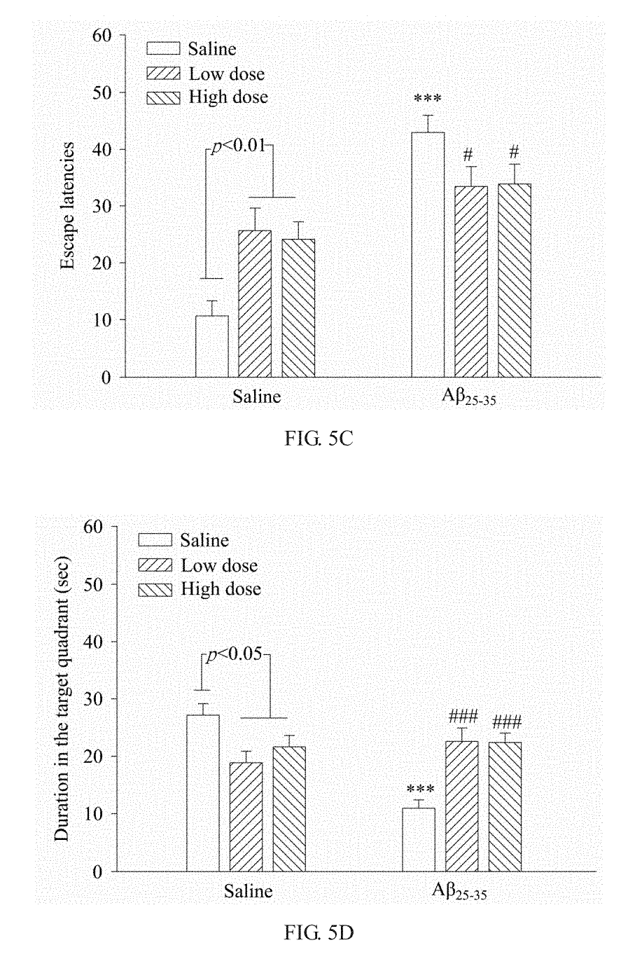

Subsequently, the influence caused by treating with MGCD0103 against the deterioration of spatial learning and long-term memory ability caused by oligomeric Aβ₂₅₋₃₅ is observed. In the embodiments of the present invention, the spatial learning and long-term memory ability are evaluated by the Morris Water Maze (MWM). First, the mice are placed in a pool filled with milky white non-toxic advertising pigment (used to make the water become opaque to hide the platform so that mice are unable to see the position of the platform at the beginning), and are allowed to explore the platform (fixed in a predetermined quadrant) underwater. FIGS. 5A to 5D show the analysis results of MWM. According to the results of FIG. 5A, which illustrate that mice of different groups all swim at a same velocity in the water maze, that is, their innate body strengths are the same. FIG. 5B shows the learning curve of the mice performing in a training trial in a period of 4 days. Accordingly, injecting saline into the brain of normal mice does not affect their learning ability so that the results show an effective curve (α3); the time required to reach the platform of the mice administrated with the low dose (▾) or the high dose (Δ) of MGCD0103 into the hippocampal CA1 thereof does not significantly decrease with an increasing number of training days. When the oligomer Aβ₂₅₋₃₅ is injected into mouse brain, it may cause a significantly decrease in learning ability so that a non-effective curve (●) is shown; whereas the mice treated with the low dose (▪) or the high dose of MGCD0103 within the hippocampal CA1 injected with oligomer Aβ₂₅₋₃₅ may experience a reduced time required to reach the platform depending on the increased number of training days. Hence, the mice treated with the low or high dose of MGCD0103 have a learning ability curve between that of the mice treated with saline and oligomer Aβ₂₅₋₃₅, that is, the drug has potential to improving learning ability. After four days of training, the testing was performed on day 5, and the time required for arriving at the platform is recorded. The spatial learning ability of the mice was determined according to the results. As shown in FIG. 5C, the time required to reach the platform for the mice of the group injected with oligomeric Aβ₂₅₋₃₅ into the hippocampal CA1 thereof is significantly increased in comparison with that of the group injected with saline, and this shows that the oligomeric Aβ₂₅₋₃₅ has cause the deterioration in the learning ability of the mice. On the other hand, for the group treated with the low dose or high dose of MGCD0103, the time required for mice reaching the platform is significantly less. However, under the saline treatment, the time required for mice to reach the platform is also significantly increased. Thus, according to the above results, MGCD0103 may provide relief from the deterioration in learning ability caused by oligomeric Aβ₂₅₋₃₅ in mice; however, the drug MGCD0103 may also cause deterioration in the learning ability of normal mice. 24 hours after the last testing trial, the platform is removed and the time that the mice spend at the quadrant where the platform was (the target quadrant) is calculated to evaluate the long-term memory. As shown in FIG. 5D the group injected with oligomeric Aβ₂₅₋₃₅ has a significantly lower time (spent at the target quadrant) in comparison with that of the group injected with saline. It shows that oligomeric Aβ₂₅₋₃₅ causes deterioration to the long term spatial memory ability of mice. Whereas, after the treatment of administrating the low dose or high dose MGCD0103, it was found that the time (spent at the target quadrant) is significantly increased in comparison with that of the Aβ₂₅₋₃₅ group without treatment, that is, MGCD0103 may be helpful in improve spatial long-term memory. However, under the saline treatment, the time spent at the target quadrant for both groups treated with the low or high dose of MGCD0103 is significantly decreased. According to the above results, MGCD0103 may improve the spatial learning and long-term memory ability of mice injected with oligomeric Aβ₂₅₋₃₅, but may be damaging to normal mice to a certain extent.

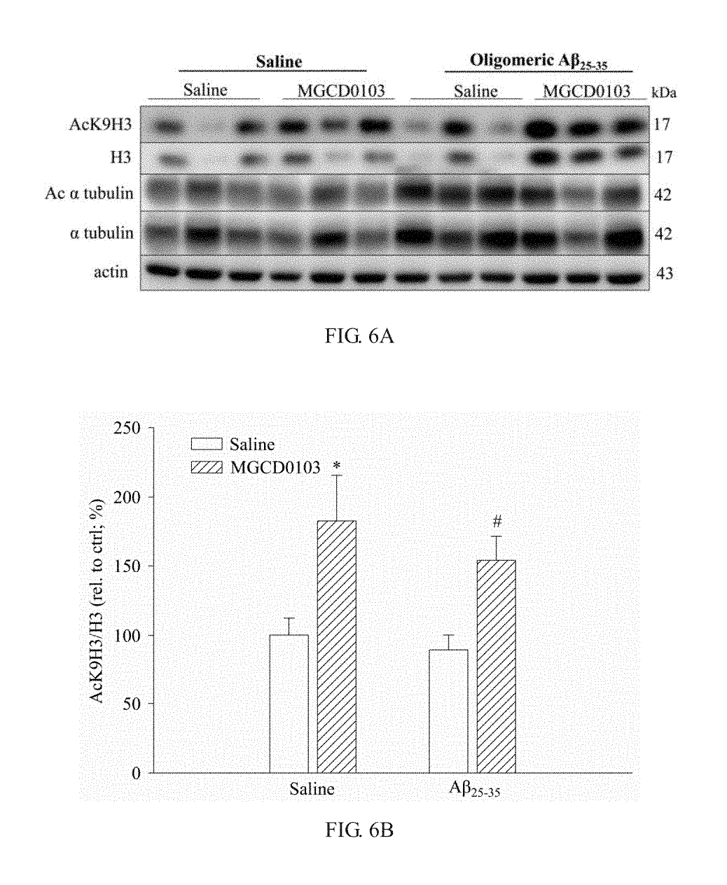

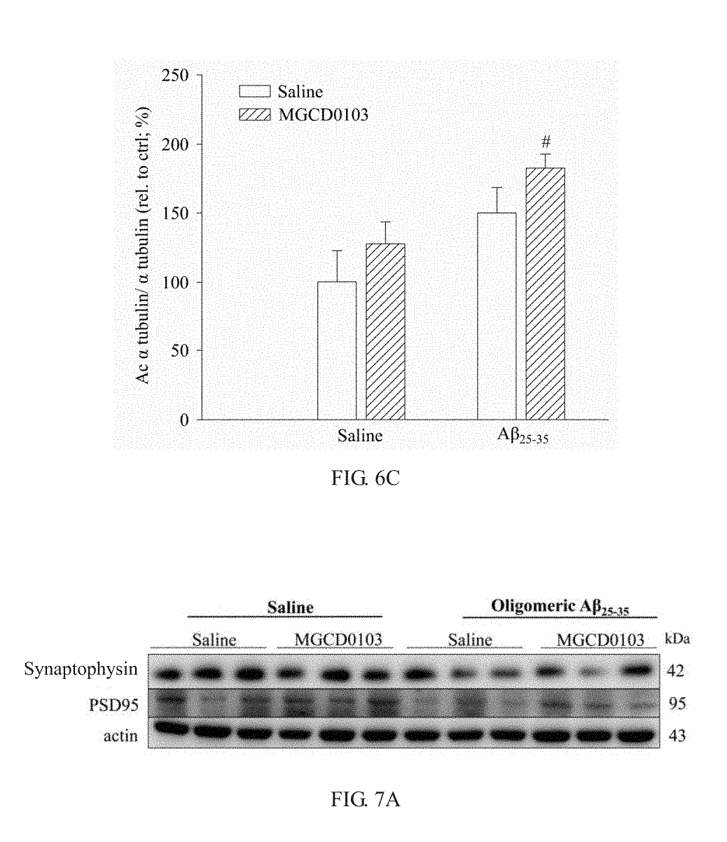

Similarly, MGCD0103 may effectively improve the acetylation level of tissue proteins H3 and alpha-tubulin. Particularly, the present invention carries out further analysis with immunoblotting analysis. As shown in FIGS. 6A to 6C, although the cells treated with oligomeric Aβ₂₅₋₃₅ may not reduce the acetylation level of tissue proteins H3 and alpha-tubulin (FIGS. 6A to 6C), MGCD0103 may significantly improve the acetylation level of tissue proteins H3 and alpha-tubulin (FIGS. 6A to 6C). These results show that acute injection of oligomeric Aβ₂₅₋₃₅ within hippocampus does not significantly affect the acetylation level of tissue proteins H3 and alpha-tubulin, but chronic treatment of MGCD0103 improves both the acetylation level of tissue proteins H3 and alpha-tubulin.

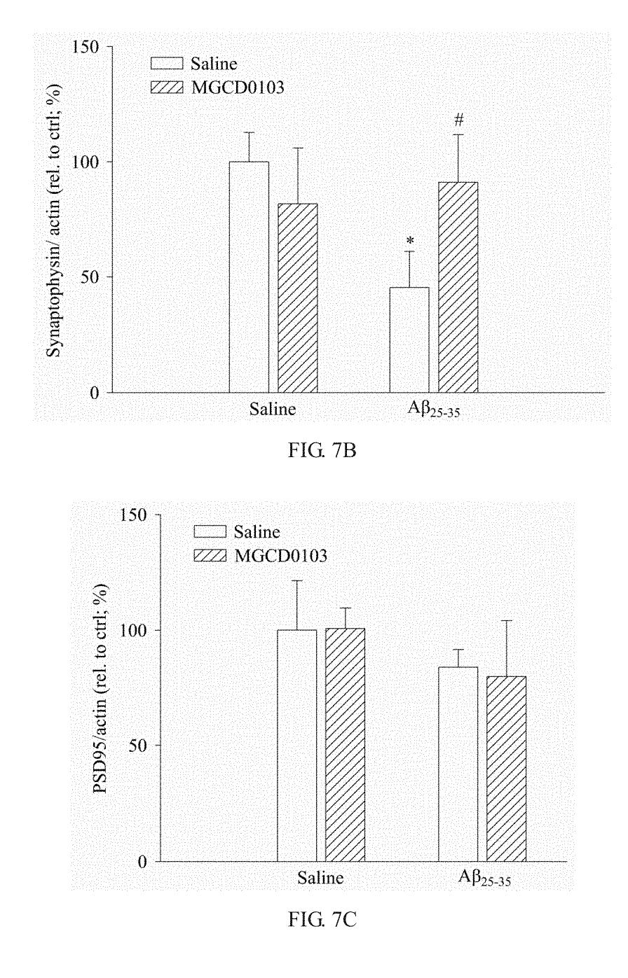

In addition, MGCD0103 may effectively improve the expression of synaptophysin. Particularly, as shown in FIGS. 7 A to 7C, the expression of synaptophysin in the CA1 injected with oligomeric Aβ₂₅₋₃₅ is significant decreased in comparison with that of the group treated with saline (FIGS. 7A and 7B). Whereas, the expression of synaptophysin in the group treated with oligomeric Aβ₂₅₋₃₅ is significantly increased after administrating MGCD0103. However, in terms of the expression of PSD95, there is no significant difference in MGCD0103 (FIGS. 7A and 7C). That is, the treatment of Aβ₂₅₋₃₅ may reduce the expression of synaptophysin, which is a functional protein in synapses, in the hippocampus; whereas the administration of MGCD0103 may improve the expression of synaptophysin.

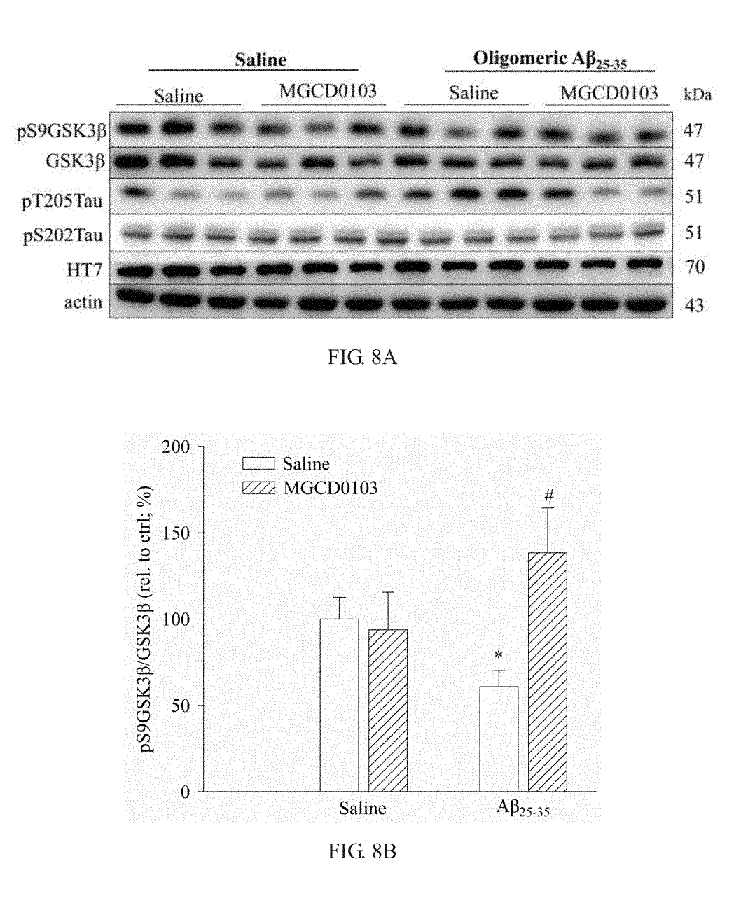

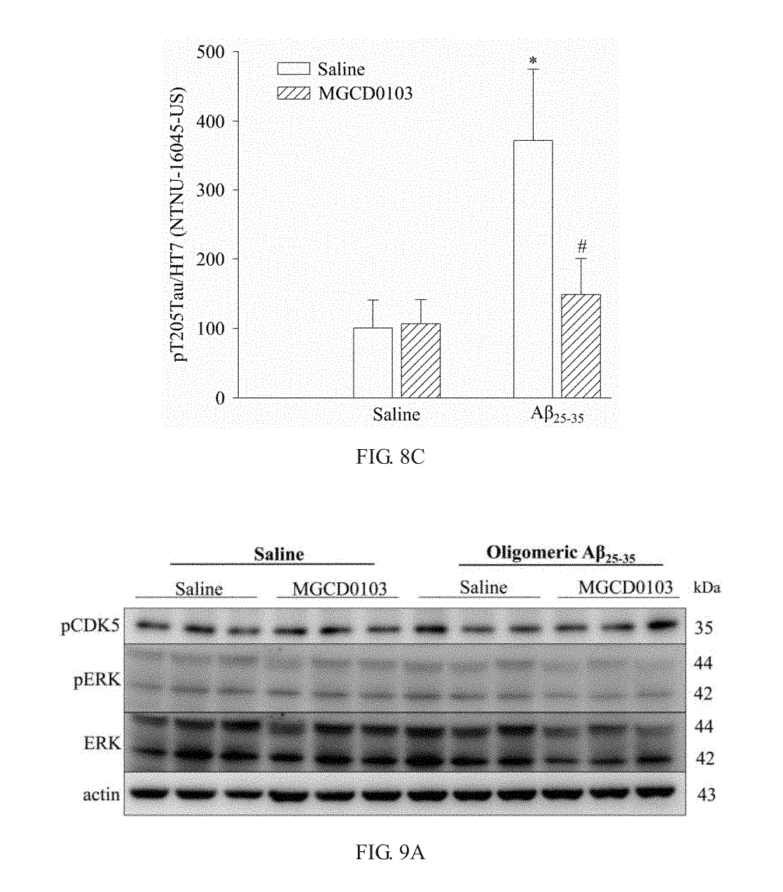

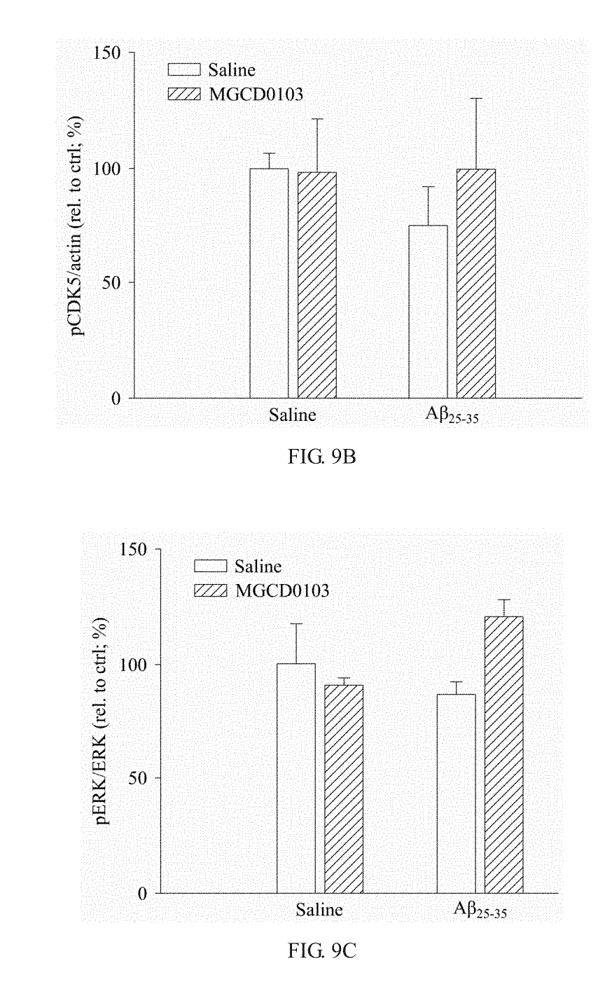

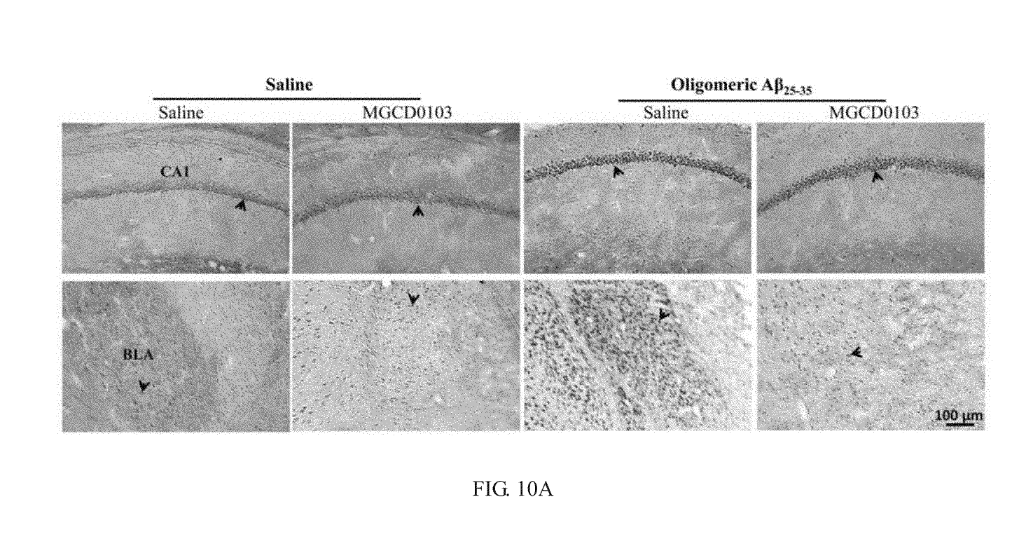

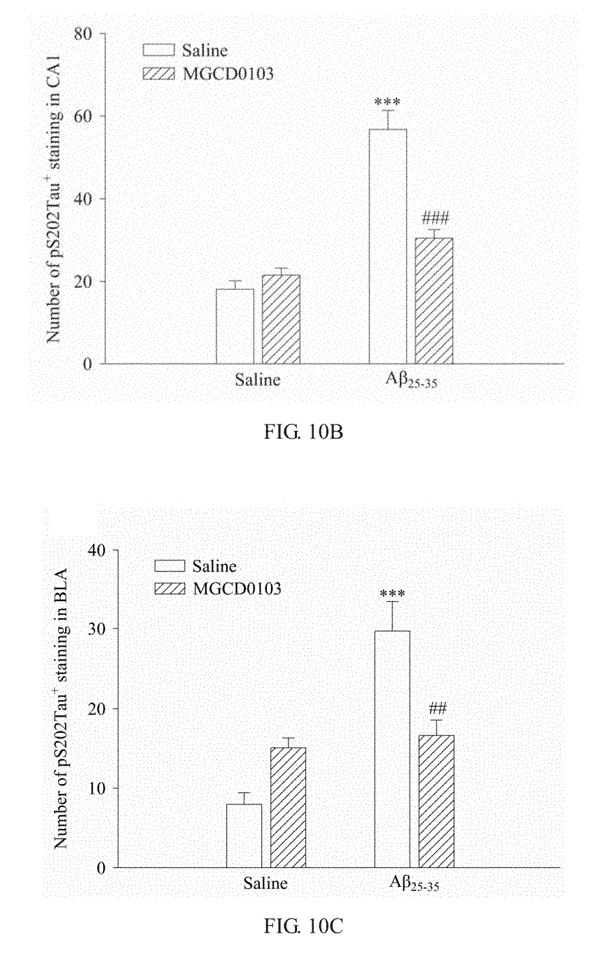

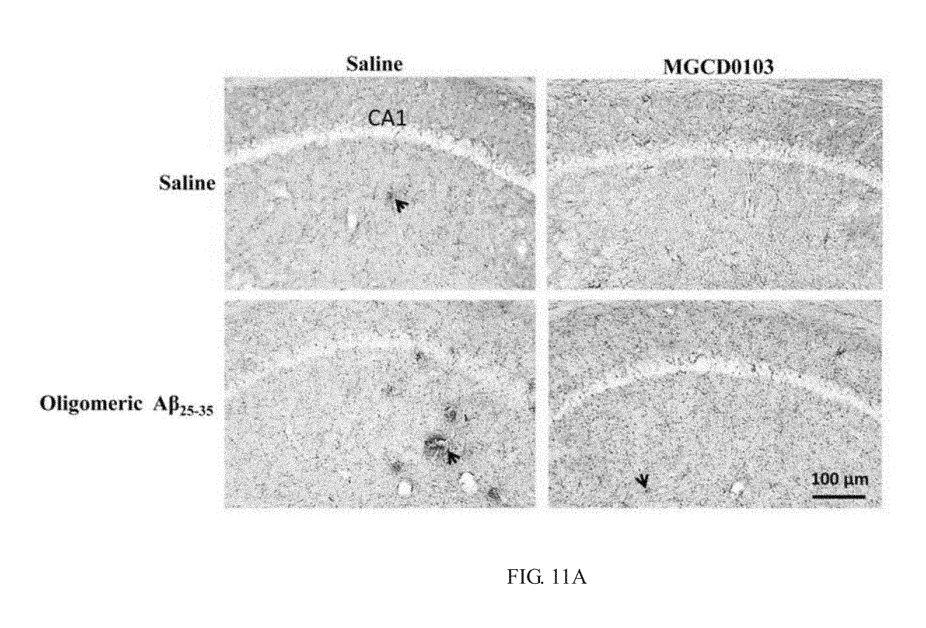

Further, MGCD0103 may effectively reduce hyperphosphorylation of the tau protein in hippocampus caused by oligomeric Aβ₂₅₋₃₅. Particularly, as shown in FIGS. 8A to 8C, the mice injected with oligomer Aβ₂₅₋₃₅ within bilateral hippocampal CA1 has a significantly decreased expression of inactivated GSK3β (pS9) enzyme; but improved hyperphosphorylation expression of Tau protein at Thr-205 site. However, when administrating MGCD0103, the expression of inactivated GSK3β (pS9) may be considerably increased, and the phosphorylation expression of Tau protein at Thr-205 is decreased. In addition, the expression of pCDK and pERK which are related to phosphorylation do not show difference between them (FIGS. 9A to 9C). According to the above results, the decrease of the phosphorylation of the Tau protein by MGCD0103 is caused by improving the expression of inactivated GSK3β (pS9) enzyme, but not by the mechanisms related to pCDK and pERK. Further, the result of phosphorylation of the Tau protein at the Ser-202 site is analyzed by an immunochemical tissue slice of the mice's hippocampus (FIGS. 10A to 10C). FIG. 10A is a stained tissue slice showing the phosphorylation of the Tau protein at the Ser-202 site in hippocampal CA1 and BLA region; and FIGS. 10B and 10C are plots illustrating the quantitative results thereof. According to the results, it was found that pS202Tau is significantly decreased after administrating MGCD0103, that is, MGCD0103 may provide relief from the hyperphosphorylation of the tau protein within the hippocampal CA1 and BLA region caused by oligomeric Aβ₂₅₋₃₅.

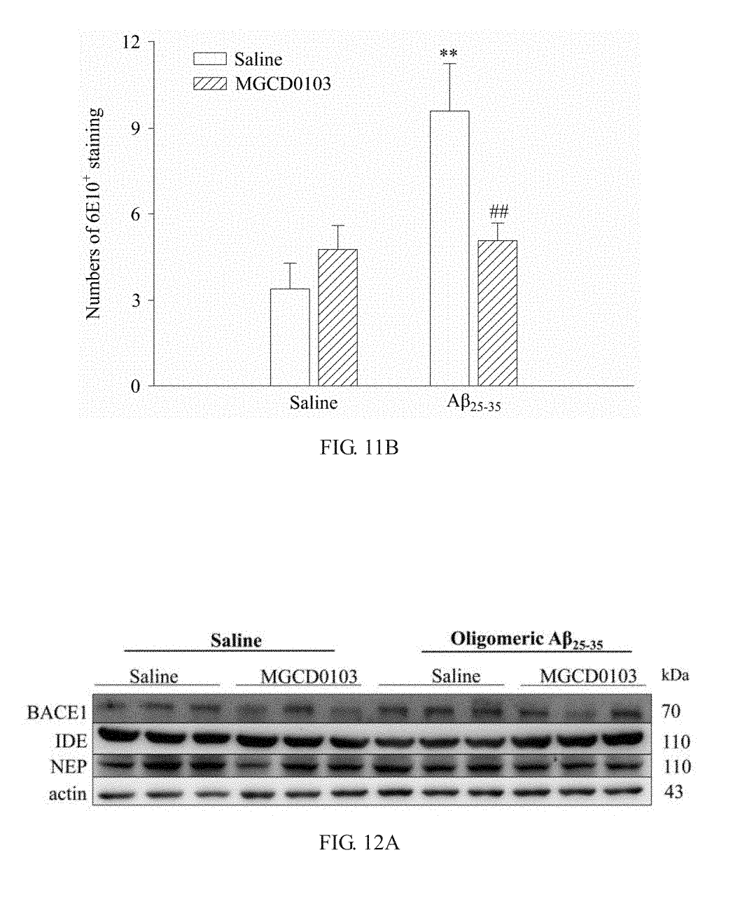

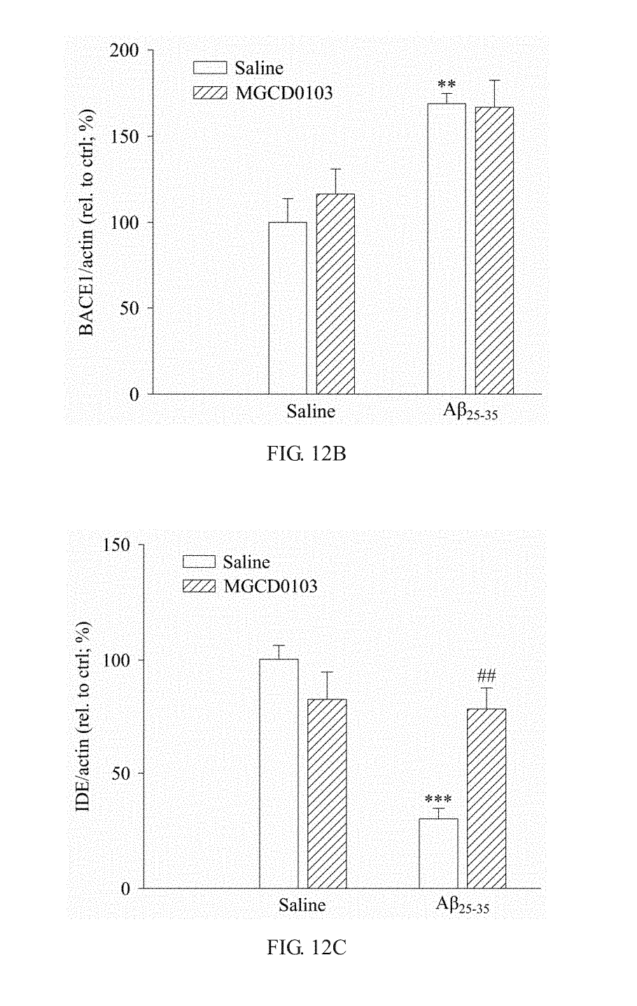

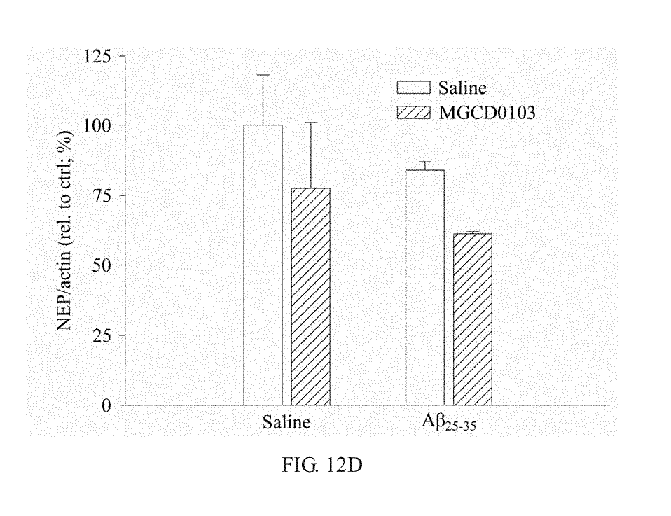

Similarly, MGCD0103 may effectively reduce accumulation of β-amyloid by improving the expression of IDE protein (Aβ degrading enzyme). As shown in FIGS. 11A and 11B, the amount of Aβ accumulated within the hippocampal CA1 is analyzed by observing the immunochemical tissue slices of the mice hippocampus. It was found that the amount of Aβ accumulated within the hippocampal CA1 of the group injected with oligomeric Aβ₂₅₋₃₅ is significantly more than the group injected with saline. Whereas, the Aβ accumulation caused by oligomeric Aβ₂₅₋₃₅ may be relieved by treatment with MGCD0103, that is, treatment with MGCD0103 may provide relief from the Aβ accumulation caused by oligomeric Aβ₂₅₋₃₅. Hence, as shown in FIGS. 12A to 12D, the present invention further analyzes the expression of BACE1 (Aβ forming enzyme), IDE (Aβ degrading enzyme) and NEP (Aβ degrading enzyme) proteins within the hippocampus by a western blot. It was found that the expression of BACE1 in the group injected with oligomeric Aβ₂₅₋₃₅ is significantly more than the group injected with saline (FIGS. 12A and 12B); but the expression of BACE1 does not show a significant difference after administrating MGCD0103; the expression of IDE in the group injected with oligomeric Aβ₂₅₋₃₅ is significantly less than that of the group injected with saline (FIGS. 12A and 12C); but the expression of IDE protein in the group injected with oligomeric Aβ₂₅₋₃₅ is significantly increased after administrating MGCD0103 (FIGS. 12A and 12D). The expression of NEP does not show a significant difference between the group injected with oligomeric Aβ₂₅₋₃₅ and the group injected with saline. Further, they still do not have significant difference in the expression of NEP after administrating MGCD0103 (FIGS. 12A and 12D). Therefore, the oligomeric Aβ₂₅₋₃₅ may increase the Aβ accumulation by improving the expression of BACE1 and reducing the expression of IDE proteins. Whereas, the MGCD0103 drug reduces the Aβ accumulation by improving the expression of IDE protein.

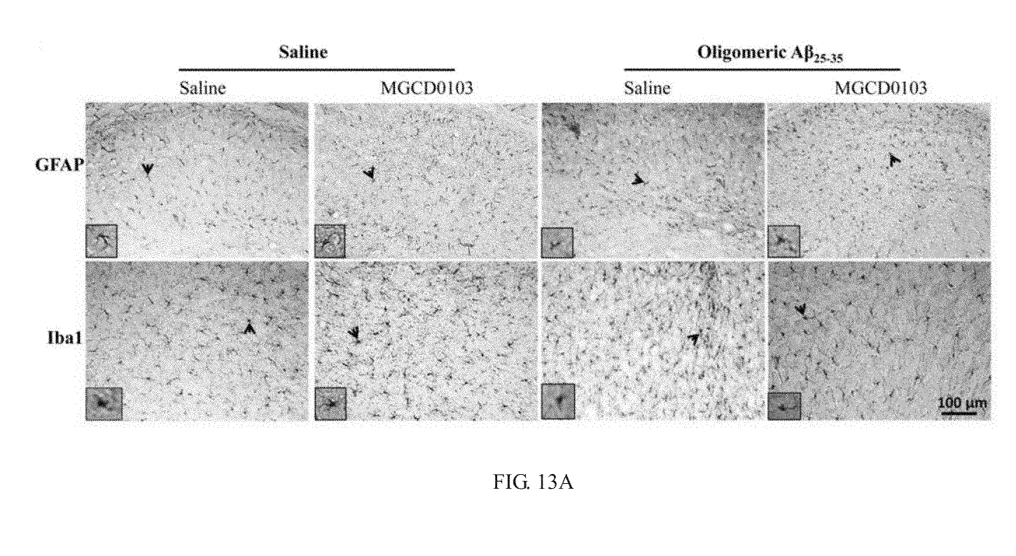

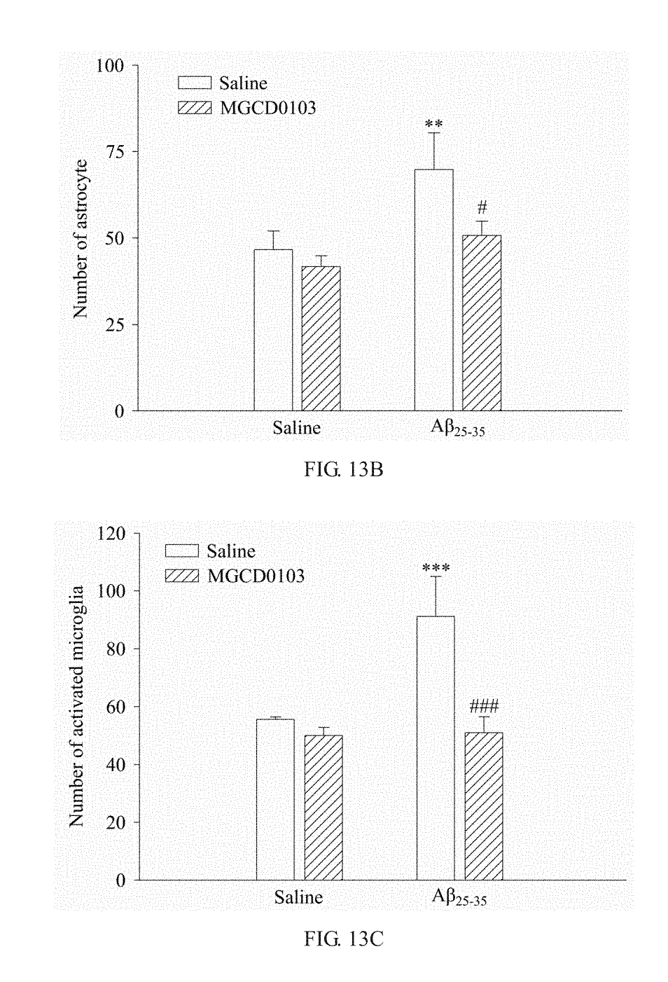

Further, MGCD0103 may provide relief from the neuroinflammation reaction caused by oligomeric Aβ₂₅₋₃₅. As shown in FIGS. 13A and 13C, the numbers of astrocytes in the mice significantly increase in the CA1 injected with oligomeric Aβ₂₅₋₃₅, whereas the numbers of astrocyte significantly decrease after administrating MGCD0103 (FIGS. 13A and 13B). Furthermore, the numbers of activated microglia significantly increase in the CA1 injected with oligomeric Aβ₂₅₋₃₅, whereas the numbers of activated microglia significantly decrease after administrating MGCD0103 (FIGS. 13A and 13C).

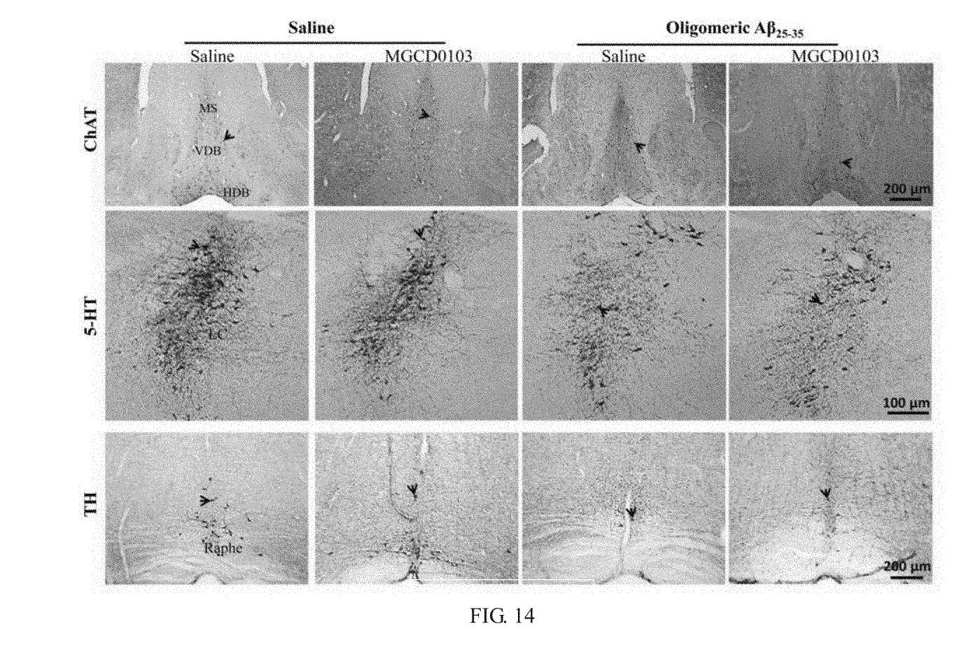

Subsequently, the inventor further observes the effect of MGCD0103 on the numbers of serotonergic neurons in Raphe nucleus, cholinergic neurons in medial septum/diagonal band (MS/DB) and adrenergic neurons in locus coeruleus (LC). As shown in FIG. 14, the present invention observes the numbers of serotonergic neurons, cholinergic neurons and adrenergic neurons. The quantitative results thereof are shown in Table 1 as follows:

TABLE 1

| | Saline | | Oligomeric Aβ₂₅₋₃₅ | |
|---|---|---|---|---|
| | Saline | MGCD0103 | Saline | MGCD0103 |
| ChAT | 69.17 ± 7.71 | 52.75 ± 8.27 | 51.00 ± 1.93 | 56.25 ± 6.78 |
| 5HT | 20.17 ± 1.32 | 21.43 ± 1.32 | 9.33 ± 2.59 ↓↓↓^{a} | 15.75 ± 1.19 ↑^{b} |
| TH | 51.50 ± 10.85 | 44.58 ± 2.05 | 21.81 ± 1.80 ↓↓↓^{a} | 24.38 ± 0.24 |

^{a}represents a comparison with the group injected with saline;
^{b}represents a comparison with the group treated with Aβ₂₅₋₃₅ alone;
↑ represents increase (p < 0.05);
↓↓↓ represents decrease (p < 0.001)

Referring to FIG. 14 and Table 1, they show that the numbers of serotonergic neurons and adrenergic neurons are significantly less in the group injected with oligomeric Aβ₂₅₋₃₅ compared with the group injected with saline. Whereas, the number of serotonergic neurons significantly increases after administrating MGCD0103, that is, the groups treated with MGCD0103 may provide relief from the decreasing of the numbers of serotonergic caused by oligomeric Aβ₂₅₋₃₅ without affecting the numbers of cholinergic neurons and adrenergic neurons.

According to the above results, MGCD0103 as used in the present invention is shown to be effective in protecting neurons in the in vitro and the in vivo experiments. Further, according to the in vivo experiments, administrating MGCD0103 may reduce the accumulation of β-amyloid, hyperphosphorylation of the tau protein, and neuroinflammation, and may increase the numbers of serotonergic neurons and the expression of synaptophysin protein. Hence, it may be an effective solution providing relief from increased levels of anxiety, and the deterioration of learning ability and the short and long term memory loss caused by the oligomer Aβ₂₅₋₃₅.

In summary, the invention disclosed herein has been described by means of exemplary embodiments and appended drawings. However, numerous modifications, variations and enhancements can be made thereto by those skilled in the art without changing the essential characteristics or technical spirit of the present invention. Therefore, it is to be understood that the present invention is not limited to the forms described in the exemplary embodiments and appended drawings, rather that the technical and protective scope of the present invention is defined by the following claims.

1. A method for enhancing learning ability and memory of patients with Alzheimer's disease using mocetinostat (MGCD0103), wherein
an effective dose of mocetinostat is administered into a subject in need.

2. The method as in claim 1, wherein the effective dose is 0.01˜2 mg per kilogram of body weight.

3. The method as in claim 1, wherein mocetinostat is a prodrug thereof, a solution thereof, or any combination thereof.

4. The method as in claim 1, wherein mocetinostat is used in combination with a pharmaceutically acceptable excipient.

5. The method as in claim 4, wherein mocetinostat is used in combination with saline and the pharmaceutically acceptable excipient, wherein a ratio of the saline and the pharmaceutically acceptable excipient is 3˜8:1.

6. The method as in claim 4, wherein the pharmaceutically acceptable excipient is a lipophilic excipient, a filler, a wetting agent, an adhesive agent, or a disintegrant.

7. The method as in claim 6, wherein the lipophilic excipient is Kolliphor®.

8. The method as in claim 1, wherein a route of administration comprises oral, intramuscular, subcutaneous or brain administration.

9. The method as in claim 1, wherein mocetinostat reduces an accumulation of β-amyloid (Aβ), a hyperphosphorylation of tau proteins, and neuroinflammation, and increases numbers of serotonergic neurons and an expression level of synaptophysin.
